(19) United States
(12) Patent Application Publication
(10) Pub. No.: US 2020/0129913 A1
(43) Pub. Date: Apr. 30, 2020

(54) METAL ORGANIC FRAMEWORK WITH TWO ACCESSIBLE BINDING SITES PER METAL CENTER FOR GAS SEPARATION AND GAS STORAGE

(71) Applicants: Libo Li, San Antonio, TX (US); Jinping Li, San Antonio, TX (US); Banglin Chen, San Antonio, TX (US)

(72) Inventors: Libo Li, San Antonio, TX (US); Jinping Li, San Antonio, TX (US); Banglin Chen, San Antonio, TX (US)

(21) Appl. No.: 16/666,361

(22) Filed: Oct. 28, 2019

Related U.S. Application Data
(60) Provisional application No. 62/751,540, filed on Oct. 27, 2018.

(51) Int. Cl. B01D 53/02; C07C 7/12; C07F 15/02

(52) U.S. Cl. CPC B01D 53/02; C07C 7/12; C07C 11/04; B01D 2253/204; C07F 15/025

(57) ABSTRACT

The separation of ethane from its corresponding ethylene is a very important, challenging and energy-intensive process in the chemical industry. Herein we report a microporous metal-organic framework Fe₂(O₂)(dobdc) (dobdc⁴⁻: 2,5-dioxido-1,4-benzenedicarboxylate) with Fe-peroxo sites for the preferential binding of ethane over ethylene and thus highly selective separation of C₂H₆/C₂H₄. Neutron powder diffraction studies and theoretical calculations demonstrate the key role of Fe-peroxo sites for the recognition of ethane. The high performance of Fe₂(O₂)(dobdc) for the ethane/ethylene separation has been validated by gas sorption isotherms, ideal adsorbed solution theory calculations, simulated and experimental breakthrough curves. Through a fixed-bed column packed with this porous material, polymer-grade of ethylene (99.99%) can be straightforwardly produced from ethane/ethylene mixtures during the first adsorption cycle, demonstrating its enormous potential for this very important industrial separation with low energy cost

### CROSS-REFERENCE TO RELATED APPLICATIONS

This application claims the benefit under Title 35 United States Code § 119(e) of U.S. Provisional Patent Application Ser. No. 62/751,540; Filed: Oct. 27, 2018, the full disclosure of which is incorporated herein by reference.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### BACKGROUND OF THE INVENTION

### I. Field of the Invention

The present disclosure relates generally to the fields of chemistry and materials science. More particularly, it concerns metal-organic frameworks, compositions thereof and methods use thereof, including for separating gas molecules such as ethylene and enthane.

### II. Description of Related Art

Ethylene (C₂H₄) is the largest feed stock in petrochemical industries with a global production capacity over 170 million tons in 2016. It is usually produced by steam cracking or thermal decomposition of ethane (C₂H₆), in which certain amount of C₂H₆ residue co-exists in the production and needs to be removed to produce polymer-grade (≥99.95%) C₂H₄ as the starting chemical for many products, particularly the widely utilized polyethylene. The well-established industrial separation technology of the cryogenic high-pressure distillation process is one of the most energy-intensive processes in chemical industry, which requires very large distillation columns with 120 to 180 trays and high reflux ratios because of the very similar sizes and volatilities of C₂H₄ and C₂H₆(1,2). Realization of cost and energy efficient C₂H₄/C₂H₆ separation to obtain polymer-grade C₂H₄ is highly desired, and has been recently highlighted as one of the most important industrial separation tasks for future energy-efficient separation processes (3-5).

Adsorbent based gas separation, either through PSA (pressure swing adsorption), TSA (temperature swing adsorption) or membranes, is a very promising technology to replace the traditional cryogenic distillation and thus to fulfill the energy-efficient separation economy. Some adsorbents such as γ-Al₂O₃(6), zeolite (7, 8), and metal-organic frameworks (MOFs) (9,10) for C₂H₄/C₂H₆ adsorptive separation have been developed. These porous materials take up larger amounts of C₂H₄ than C₂H₆, mainly due to the stronger interactions of the immobilized metal sites such as Ag (I) and Fe (II) on the pore surfaces with unsaturated C₂H₄ molecules (9, 11).

Although these kinds of adsorbents exhibit excellent adsorption separation performance towards C₂H₄/C₂H₆ mixture with the selectivity up to 48.7 (12), production of high-grade C₂H₄ is still quite energy-intensive. This is because C₂H₄, as the preferentially adsorbed gas, needs to be further desorbed to get the C₂H₄ product. In order to remove the un-adsorbed and contaminated C₂H₆, at least four adsorption-desorption cycles through inert gas or vacuum pump are necessary to achieve the purity limit required (≥99.95%) for the C₂H₄ polymerization reactor (13).

If C₂H₆ is preferentially adsorbed, the desired C₂H₄ product can be directly recovered in the adsorption cycle. Compared to those C₂H₄ selective adsorbents, it can save approximately 40% energy consumption (0.4-0.6 GJ/t ethylene) (14-15) on PSA technology for the C₂H₄/C₂H₆ separation. Although porous materials have been well established for gas separation and purification (16-22), those exhibiting the preferred C₂H₆ adsorption over C₂H₄ are scarce. To date, only a few porous materials for the selective C₂H₆/C₂H₄ separation have been reported (2, 13, 23, 24) with quite low separation selectivity and productivity.

To target MOFs with the preferential binding of C₂H₆ over C₂H₄, it is necessary to immobilize some specific sites for the stronger interactions with C₂H₆. Inspired by natural metalloenzymes and synthetic compounds for alkane C—H activation in which M-peroxo, M-hydroperoxo and M-oxo (M=Cu(II), Co (III) and Fe (III/IV)) are active catalytic intermediates (25-27), we hypothesized that similar functional sites within MOFs might have stronger binding with alkanes than alkenes, and thus can be utilized for the selective separation of C₂H₆/C₂H₄. In this regard, Fe₂(O₂)(dobdc) developed by Bloch et al. containing iron(III)-peroxo sites on the pore surfaces might be of special interest (28, 29). We thus synthesized the Fe₂(O₂)(dobdc), studied its binding for C₂H₆ and examined the separation performance for C₂H₆/C₂H₄ mixtures. Indeed, we found that Fe₂(O₂)(dobdc) exhibits preferential binding of C₂H₆ over C₂H₄. Fe₂(O₂)(dobdc) not only takes up moderately high amount of C₂H₆, but also displays the highest C₂H₆/C₂H₄ separation selectivities in the wide pressure range among the examined porous materials, demonstrating it as the best material ever reported for this very important gas separation to produce polymer-grade ethylene (99.99%).

### SUMMARY OF THE INVENTION

In some aspects, the present disclosure provides MOFs which may be used to remove one type of molecules from a mixture. In some aspects the MOF is a microporous metal-organic framework Fe2(O2)(dobdc) (dobdc4-: 2,5-dioxido-1,4-benzenedicarboxylate) with Fe-peroxo sites for the preferential binding of ethane over ethylene and thus highly selective separation of C2H6/C2H4.

In some aspects, the present disclosure provides a method for separating a mixture comprising ethane and ethylene comprising:

Contacting the mixture with a microporous metal-organic framework (MOF) with Fe-peroxo sites wherein that MOF has a binding preference for ethane over ethylene.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The invention may be better understood by reference to one of these drawings in combination with the detailed description of specific embodiments presented herein.

FIGS. 1A-1C show Structures of (A) Fe₂(dobdc), (B) Fe₂(O₂)(dobdc) and (C) Fe₂(O₂)(dobdc)⊃C₂D₆ at 7 K, determined from neutron powder diffraction studies. Note the change from the open Fe(II) site to Fe(III)-peroxo site for the preferential binding of ethane. (Fe, green; C, dark grey; O, pink; O₂²⁻, red; H or D, white; C in C₂D₆, blue).

FIGS. 2A-2F shows C₂H₆ and C₂H₄ adsorption isotherms of Fe₂(O₂)(dobdc), IAST calculations and separation potentials simulations on C₂H₆ selective MOFs. (A) Adsorption (solid) and desorption (open) isotherms of C₂H₆ (red circles) and C₂H₄ (blue circles) in Fe₂(O₂)(dobdc) at 298 K. (B and C) Comparison of the IAST selectivities of Fe₂(O₂)(dobdc) versus those of previously reported best-performing materials for C₂H₆/C₂H₄ (50/50 and 10/90) mixtures. (D) Predicted productivity of 99.95% pure C₂H₄ from C₂H₆/C₂H₄ (50/50 and 10/90) mixtures in fixed bed adsorbers at 298 K. (E and F) Separation potential of Fe₂(O₂)(dobdc) for C₂H₆/C₂H₄ (50/50 left and 10/90 right) mixtures versus best-performing MOFs.

FIGS. 3A-3D shows experimental column breakthrough curves for (A) C₂H₆/C₂H₄ (50/50) mixture, (B) cycling test of C₂H₆/C₂H₄ (50/50) mixtures, (C) C₂H₆/C₂H₄(10/90) mixtures and (D) C₂H₆/C₂H₄/C₂H₂/CH₄/H₂ (10/87/1/1/1) mixtures in an absorber bed packed with Fe₂(O₂)(dobdc) at 298 K and 1.01 bar.

### DESCRIPTION OF ILLUSTRATIVE EMBODIMENTS

All of the compounds, compositions, and methods disclosed and claimed herein can be made and executed without undue experimentation in light of the present disclosure. While the disclosure may have focused on several embodiments or may have been described in terms of preferred embodiments, it will be apparent to those of skill in the art that variations and modifications may be applied to the compounds, compositions, and methods without departing from the spirit, scope, and concept of the invention. All variations and modifications apparent to those skilled in the art are deemed to be within the spirit, scope, and concept of the invention as defined by the appended claims.

Fe₂(O₂)(dobdc) was prepared according to the previously reported procedure with a slight modification (28).

### Synthesis of Fe₂(dobdc)

Anhydrous ferrous chloride (0.33 g, 2.7 mmol), 2,5-dihydroxyterephthalic acid (0.213 g, 1.08 mmol), anhydrous DMF (50 mL), and anhydrous methanol (6 mL) were added to a 100 mL three-neck flask in glove box filled with 99.999% N₂. The reaction mixture was heated to 393 K and stirred for 18 h to form red-orange precipitate. Methanol exchange was repeated six times during 2 days, and the solid was collected by filtration and dry in vacuum to yield Fe₂(dobdc)·solvent as a yellow-ochre powder. Fe₂(dobdc)·solvent sample was fully activated by heating under dynamic vacuum (<10⁻⁷ bar) at 433 K for 18 h and then cooled down to room temperature to yield Fe₂(dobdc) as light green powder (28). Fe₂(dobdc) is air-sensitive, so needs to be handled and stored in a dry box under N₂ atmosphere.

### Synthesis of Fe₂(O₂)(dobdc)

Fe₂(O₂)(dobdc) was synthesized under carefully controlled conditions (28): About 1.3 g Fe₂(dobdc) sample was transferred into a 500 mL flask in dry glove box, then sealed and evacuated to 10⁻⁷ bar. Pure O₂ (>99.999%) was slowly dosed to the bare Fe₂(dobdc) sample to 0.01 bar at a rate of 0.5 mbar/min under 298 K, then the O₂ pressure was brought up to 1 bar and the sample was allowed to sit for 1 h to reach equilibrium. At last, the sample was fully evacuated under high vacuum, and the free O₂ gas molecules in the pore channels were completely removed to yield Fe₂(O₂)(dobdc) as dark brown powder. Fe₂(O₂)(dobdc) is air-sensitive, so needs to be handled and stored in a dry box under N₂ atmosphere.

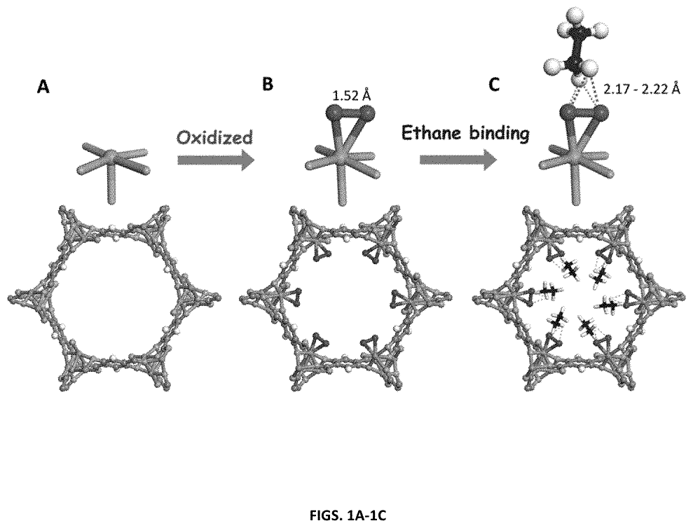

Both Fe₂(dobdc) and Fe₂(O₂)(dobdc) are air-sensitive, and need to be handled and stored in a dry box under N₂ atmosphere. As expected, Fe₂(O₂)(dobdc) maintains the framework structure of Fe₂(dobdc) (FIGS. 1A, 1B, and S1A), with a BET (Brunauer-Emmett-Teller) surface area of 1073 m²/g (FIG. S1B).

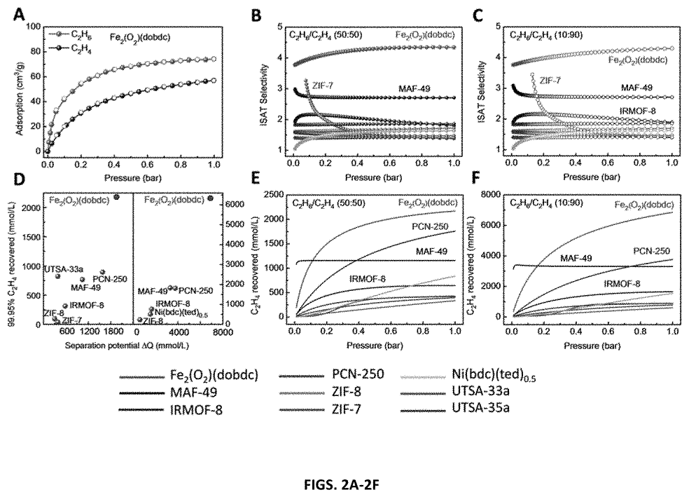

The C₂H₆ binding affinity in Fe₂(O₂)(dobdc) was first investigated by single-component sorption isotherms at temperature of 298 K and pressure up to 1 bar, as shown in FIG. 2A. The C₂H₆ adsorption capacity on Fe₂(O₂)(dobdc) is much higher than that of C₂H₄, implying its unique binding affinity for C₂H₆. At 1 bar, the uptake amount of C₂H₆ in Fe₂(O₂)(dobdc) is 74.3 cm³/g, corresponding to ˜1.1 C₂H₆ per Fe₂(O₂)(dobdc) formula. Unlike the pristine Fe₂(dobdc), which takes up more C₂H₄ than C₂H₆ because of the Fe(II) open sites, Fe₂(O₂)(dobdc) adsorbs larger amount of C₂H₆ than C₂H₄. Therefore we successfully realized the “reversed C₂H₆/C₂H₄ adsorption” in Fe₂(O₂)(dobdc) (FIG. S2). The adsorption heat (Qₛₜ) of C₂H₆ and C₂H₄ on Fe₂(O₂)(dobdc) were calculated by using the Virial equation (FIG. S3). The C₂H₆ adsorption heat of Fe₂(O₂)(dobdc) was calculated to be 66.8 kJ/mol at zero coverage, a much higher value than the reported ones of other MOFs (2), indicating the strong interaction between Fe₂(O₂)(dobdc) and C₂H₆ molecules. And all of the isotherms are completely reversible and exhibit no hysteresis. Further adsorption cycling tests at 298 K (FIG. S4) indicate no loss of C2 uptake capacity over 20 adsorption/desorption cycles.

To structurally elucidate how C₂H₆ and C₂H₄ are adsorbed in this MOF, high-resolution neutron powder di raction (NPD) measurements were carried out on C₂D₆-loaded and C₂D₄-loaded samples of Fe₂(O₂)(dobdc) at 7 K (see supplementary materials and FIG. S5). As shown in FIG. 1C, C₂D₆ molecules exhibit preferential binding with the peroxo sites through C-D . . . O hydrogen bonds (D . . . O, ˜2.17-2.22 Å). The D . . . O distance is much shorter than the sum of van der Waals radii of oxygen (1.52 Å) and hydrogen (1.20 Å) atoms, indicating a relatively strong interaction, which is consistent with the high C₂H₆ adsorption heat found in Fe₂(O₂)(dobdc). In addition, we noticed that, sterically, the non-planer C₂D₆ molecule happens to match better to the uneven pore surface in Fe₂(O₂)(dobdc) than the planar C₂D₄ molecule (FIG. S6), resulting in stronger hydrogen bonds with the Fe-peroxo active site and stronger van der Waals interactions with the ligand surface. To further understand the mechanism of the selective C₂H₆/C₂H₄ adsorption in Fe₂(O₂)(dobdc), we conducted detailed first-principles dispersion-corrected density functional theory (DFT-D) calculations (see supplementary materials and table S1). The optimized C₂H₆ binding configuration on the Fe-peroxo site agrees reasonably well with the C₂D₆-loaded structures determined from the NPD data, supporting that the reversed C₂H₆/C₂H₄ adsorption selectivity originates from the peroxo active sites and the electronegative surface oxygen distribution in Fe₂(O₂)(dobdc). Interestingly, similar preferential binding of C₂H₆ over C₂H₄ has also been experimentally found in another oxidized MOF Cr-BTC(O₂) (FIGS. S7 and S8) (30).

Ideal adsorbed solution theory (IAST) calculations were performed to estimate the adsorption selectivities of C₂H₆/C₂H₄ (50/50 and 10/90) for Fe₂(O₂)(dobdc) and other C₂H₆ selective materials (FIG. 2B). The fitting details were provided in the supplementary materials (FIGS. S9-S17 and tables S2-S11). Compared to other top-performing MOFs (MAF-49, IRMOF-8, ZIF-8, ZIF-7, PCN-250, Ni(bdc)(ted)_{0.5}, UTSA-33a, and UTSA-35a), Fe₂(O₂)(dobdc) exhibits a new benchmark for C₂H₆/C₂H₄ (50/50) adsorption selectivity (4.4) at 1 bar and 298 K, greater than the previously reported best-performing MOF MAF-49 (2.7) (2). It is worth noting that this value is also higher than the highest value (2.9) of 300000 all-silica zeolite structures, which was investigated by Kim et al. through computational screening (31). For C₂H₆/C₂H₄(10/90) mixture, under the same condition, Fe₂(O₂)(dobdc) also exhibits the highest adsorption selectivity among these MOFs (FIG. 2C).

Next, transient breakthrough simulations were conducted to validate the feasibility of using Fe₂(O₂)(dobdc) in a fixed-bed for separation of C₂H₆/C₂H₄ mixtures (FIG. S18). Two C₂H₆/C₂H₄ mixtures (50/50 and 10/90) were used as feeds to mimic the industrial process conditions. The simulated breakthrough curves shows C₂H₆/C₂H₄ (50/50) mixtures were complete separated by Fe₂(O₂)(dobdc), whereby C₂H₄ breakthrough occurred first within seconds to yield the polymer-grade gas, and then C₂H₆ passed through the fixed-bed after a certain time (τ_{break}). To make an evaluation on the C₂H₆/C₂H₄ separation ability of these MOFs, separation potential ΔQ was calculated to quantify the mixture separations in fixed bed adsorbers (table S12). Attributed to the record-high C₂H₆/C₂H₄ selectivity and relatively high C₂H₆ uptake, the amount of 99.95% pure C₂H₄ recovered by Fe₂(O₂)(dobdc) reaches up to 2172 mmol/liter (C₂H₆/C₂H₄ 50/50) and 6855 mmol/liter (C₂H₆/C₂H₄ 10/90) (FIG. 2D), respectively, which are almost two times higher than the other benchmark materials. Fe₂(O₂)(dobdc) has the highest separation potential for recovering the pure C₂H₄ from (50/50) C₂H₆/C₂H₄ mixtures during the adsorption process (FIG. 2E). Even when the concentration of C₂H₆ decreases to 10% (FIG. 2F), Fe₂(O₂)(dobdc) can still keep the highest separation potential (table S13), which makes it as the most promising material for separation C₂H₆ from C₂H₆/C₂H₄ mixtures.

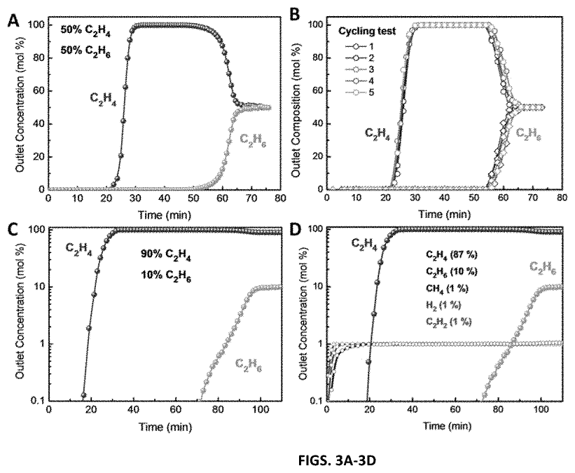

These excellent breakthrough results from simulation encouraged us to further evaluate the separation performance of Fe₂(O₂)(dobdc) in the actual separation process. Several breakthrough experiments were performed on an in-house-constructed apparatus, which was reported in our previous work (32). The breakthrough experiments were performed on several selected MOFs including Fe₂(O₂)(dobdc), in which C₂H₆/C₂H₄ (50/50) mixtures were flowed over a packed bed with a total flow rate of 5 mL/min at 298 K (FIG. S19 and table S14). For Fe₂(O₂)(dobdc), a clean and sharp separation of C₂H₆/C₂H₄ was observed (FIG. 3A). C₂H₄ was first to elute through the bed before it was contaminated with the undetectable amounts of C₂H₆, resulting in a high concentration of C₂H₄ feed to be ≥99.99% (the detection limit of the instrument is 0.01%). After some period, the adsorbent got saturated, C₂H₆ broke through, then the outlet gas stream quickly reached equimolar concentrations. To make the systematical comparison for the C₂H₄ separation performance in the selected MOFs, C₂H₄ purity and productivity were calculated from their breakthrough curves (table S15). For Fe₂(O₂)(dobdc), 0.79 mmol/g of C₂H₄ with 99.99%+ purity can be recovered from the C₂H₄/C₂H₆(50/50) mixture in a single breakthrough operation, this value is nearly three times of the benchmark material MAF-49 (0.28 mmol/g). In addition, the cycle and regeneration capabilities of Fe₂(O₂)(dobdc) were further studied by breakthrough cycle experiments (FIG. 3B), there is no noticeable decreasing in the mean residence time for both C₂H₆ and C₂H₄ within continuous 5 cycles under ambient conditions. Moreover, Fe₂(O₂)(dobdc) materials retained its stability after the breakthrough cycling test (FIG. S20).

In the real production of high-purity C₂H₄, C₂H₆ concentration in C₂H₄/C₂H₆ mixtures produced by naphtha cracking is about 6-10%, and the feed gases are also contaminated by low levels of impurities such as CH₄, H₂, and C₂H₂(33). Therefore, breakthrough experiments on C₂H₆/C₂H₄(10/90) mixtures and C₂H₆/C₂H₄/CH₄/H₂/C₂H₂(10/87/1/1/1) mixtures were also performed for Fe₂(O₂)(dobdc). As shown in FIGS. 3C and 3D, highly efficient separations for both mixtures were realized, which further demonstrate that Fe₂(O₂)(dobdc) can be used to purify the C₂H₄ with low concentration of C₂H₆ even in the presence of CH₄, H₂, and C₂H₂ impurities. In summary, we discovered that a unique metal-organic framework with Fe-peroxo sites can induce the stronger interactions with C₂H₆ than C₂H₄, leading to the unusual “reversed C₂H₆/C₂H₄ adsorption”. The fundamental binding mechanism of Fe₂(O₂)(dobdc) for the recognition of C₂H₆ has been demonstrated through neutron di raction studies and theoretical calculations, indicating the important role of the Fe-peroxo sites for the preferential interactions with C₂H₆. This material can readily produce high purity C₂H₄ (≥99.99%) from C₂H₄/C₂H₆ mixture during the first breakthrough cycle with the moderately high productivity and low energy cost. The strategy we developed here might be broadly applicable, which will facilitate the extensive research on the immobilization of different sites into porous MOFs for their stronger interactions with C₂H₆ than C₂H₄, thus targeting some practically useful porous materials with low material cost and high productivity for the practical industrial realization of this very challenging and important separation.

### REFERENCES

The following references, to the extent that they provide exemplary procedural or other details supplementary to those set forth herein, are specifically incorporated herein by reference.
1. Bander, F. Separation Technologies. http://seperationtechnology.com (2012).
2. P.-Q. Liao, W.-X. Zhang, J.-P. Zhang, X.-M. Chen, Nat. Commun. 6, 8697 (2015).
3. D. S. Sholl, R. P. Lively, Nature 532, 435-437 (2016).
4. J. Y. S. Lin, Science 353, 121-122 (2016).
5. S. Chu, Y. Cui, N. Liu, Nat. Mater. 16, 16-22 (2017).
6. R. T. Yang, E. S. Kikkinides, AIChE J. 41, 509-517 (1995).
7. P. J. Bereciartua, Á. Cantín, A. Corma, J. L. Jordá, M. Palomino, F. Rey, S. Valencia, E. W. Corcoran Jr., P. Kortunov, P. I. Ravikovitch, A. Burton, C. Yoon, Y. Wang, C. Paur, J. Guzman, A. R. Bishop, G. L. Casty, Science 358, 1068-1071 (2017).
8. S. Aguado, G. Bergeret, C. Daniel, D. Farrusseng, J. Am. Chem. Soc. 134, 14635-14637 (2012).
9. E. D. Bloch, W. L. Queen, R. Krishna, J. M. Zadrozny, C. M. Brown, J. R. Long, Science 335, 1606-1610 (2012).
10. Y. He, R. Krishna, B. Chen, Energy Environ. Sci. 5, 9107-9120 (2012).
11. B. Li, Y. Zhang, R. Krishna, K. Yao, Y. Han, Z. Wu, D. Ma, Z. Shi, T. Pham, B. Space, J. Liu, P. K. Thallapally, J. Liu, M. Chrzanowski, S. Ma, J. Am. Chem. Soc. 136, 8654-8660 (2014).
12. S. Yang, A. J. Ramirez-Cuesta, R. Newby, V. Garcia-Sakai, P. Manuel, S. K. Callear, S. I. Campbell, C. C. Tang, M. Schroder, Nat. Chem. 7, 121-129 (2015).
13. C. Gücüyener, J. van den Bergh, J. Gascon, F. Kapteijn, J. Am. Chem. Soc. 132, 17704-17706 (2010).
14. A. Mersmann, B. Fill, R. Hartmann, S. Maurer, Chem. Eng. Technol. 23, 937-944 (2000).
15. T. Ren, M. Patel, K. Blok, Energy. 31, 425-451 (2006).
16. H. Furukawa, K. E. Cordova, M. O'Keeffe, O. M. Yaghi, Science 341, 1230444 (2013).
17. H. Sato, W. Kosaka, R. Matsuda, A. Hori, Y. Hijikata, R. V. Belosludov, S. Sakaki, M. Takata, S. Kitagawa, Science 343, 167-170 (2014).
18. A. Cadiau, K. Adil, P. M. Bhatt, Y. Belmabkhout, M. Eddaoudi, Science 353, 137-140 (2016).
19. R. Vaidhyanathan, S. S. Iremonger, G. K. H. Shimizu, P. G. Boyd, S. Alavi, T. K. Woo, Science. 330, 650-653 (2010).
20. P. Nugent, Y. Belmabkhout, S. D. Burd, A. J. Cairns, R. Luebke, K. Forrest, T. Pham, S. Ma, B. Space, L. Wojtas, M. Eddaoudi, M. J. Zaworotko, Nature 495, 80-84 (2013).
21. Q.-G. Zhai, X. Bu, C. Mao, X. Zhao, L. Daemen, Y. Cheng, A. J. Ramirez-Cuesta, P. Feng, Nat. Commun. 7, 13645 (2016).
22. K. Li, D. H. Olson, J. Seidel, T. J. Emge, H. Gong, H. Zeng, J. Li, J. Am. Chem. Soc. 131, 10368-10369 (2009).
23. W. Liang, F. Xu, X. Zhou, J. Xiao, Q. Xia, Y. Li, Z. Li, Chem. Eng. Sci. 148, 275-281 (2016).
24. Y. Chen, Z. Qiao, H. Wu, D. Lv, R. Shi, Q. Xia, J. Zhou, Z. Li, Chem. Eng. Sci. 175, 110-117 (2018).
25. D. J. Xiao, E. D. Bloch, J. A. Mason, W. L. Queen, M. R. Hudson, N. Planas, J. Borycz, A. L. Dzubak, P. Verma, K. Lee, F. Bonino, V. Crocellà, J. Yano, S. Bordiga, D. G. Truhlar, L. Gagliardi, C. M. Brown, J. R. Long, Nat. Chem. 6, 590-595 (2014).
26. Z. Li, A. W. Peters, A. E. Platero-Prats, J. Liu, C.-W. Kung, H. Noh, M. R. DeStefano, N. M. Schweitzer, K. W. Chapman, J. T. Hupp, O. K. Farha, J. Am. Chem. Soc. 139, 15251-15258 (2017).
27. J. Cho, S. Jeon, S. A. Wilson, Lei. Liu, Eun A. Kang, J. J. Braymer, M. H. Lim, B. Hedman, K. O. Hodgson, J. S. Valentine, E. I. Solomon, W. Nam, Nature 478, 502-505 (2011).
28. E. D. Bloch, L. J. Murray, W. L. Queen, S. Chavan, S. N. Maximoff, J. P. Bigi, R. Krishna, V. K. Peterson, F. Grandjean, G. J. Long, B. Smit, S. Bordiga, C. M. Brown, J. R. Long, J. Am. Chem. Soc. 133, 14814-14822 (2011).
29. W. L. Queen, E. D. Bloch, C. M. Brown, M. R. Hudson, J. A. Mason, L. J. Murray, A. J. Ramirez-Cuesta, V. K. Peterson and Jeffrey R. Long, Dalton Trans. 41, 4180-4187 (2012).
30. L. J. Murray, M. Dinca, J. Yano, S. Chavan, S. Bordiga, C. M. Brown, and J. R. Long, J. Am. Chem. Soc. 132, 7856-7857 (2010).
31. J. Kim, L.-C. Lin, R. L. Martin, J. A. Swisher, M. Haranczyk, B. Smit, Langmuir 28, 11914-11919 (2012).
32. L. Li, R.-B. Lin, R. Krishna, X. Wang, B. Li, H. Wu, J. Li, W. Zhou, and B. Chen, J. Am. Chem. Soc. 139, 7733-7736 (2017).
33. R. Meyers, R. A. Meyers, Handbook of Petrochemicals Production Processes (McGraw-Hill Prof Med/Tech, 2005).
34. P. Giannozzi et al., J. Phys.: Condens. Matter 21, 395502 (2009).
35. V. Barone, M. Casarin, D. Forrer, M. Pavone, M. Sambi, A. Vittadini, J. Comput. Chem. 30, 934-939 (2009).
36. Y. He, S. Xiang, F. R. Fronczek, R. Krishna, and B. Chen, Chem. Eur. J. 18, 613-619 (2012).
37. Y. He, S. Xiang, F. R. Fronczek, R. Krishna, and B. Chen, Chem. Commun. 48, 6493-6495 (2012).

What is claimed is:

1. A method for separating a mixture comprising ethane and ethylene comprising:
contacting the mixture with a microporous metal-organic framework (MOF) with Fe-peroxo sites wherein that MOF has a binding preference for ethane over ethylene. obtaining an output stream richer in ethlyene as compared to the mixture.
